(19) United States
(12) Patent Application Publication
(10) Pub. No.: US 2025/0224352 A1
(43) Pub. Date: Jul. 10, 2025

(54) CELL COUNT AND CELL VIABILITY ASSESSMENT USING WATER PROTON NMR

(71) Applicants: UNIVERSITY OF MARYLAND, BALTIMORE, Baltimore, MD (US); Government of the United States of America, as Represented by the Secretary of Commerce, Gaithersburg, MD (US)

(72) Inventors: Yihua Bruce YU, Ellicott City, MD (US); Marc B. TARABAN, North Potomac, MD (US); Katharine T. BRIGGS, Germantown, MD (US); Zvi KELMAN, Gaithersburg, MD (US); William Bradley O'DELL, Gaithersburg, MD (US)

(21) Appl. No.: 19/006,373

(22) Filed: Dec. 31, 2024

Related U.S. Application Data
(60) Provisional application No. 63/618,427, filed on Jan. 8, 2024.

(51) Int. Cl. G01N 24/08; G01N 33/487

(52) U.S. Cl. CPC G01N 24/082; G01N 33/487

(57) ABSTRACT

A method of using the relaxation rate (R₁ and/or R₂) of solvent NMR signal to non-invasively estimate cell counts in cell cultures prepared under similar conditions as well as to use wNMR to measure cell viability. The methods can simply, rapidly, and non-invasively monitor the growth, health, and aging status of a cell culture, without any sample preparation or reagent (e.g., a dye) addition.

### CROSS REFERENCE TO RELATED APPLICATIONS

This application is filed under the provisions of 35 U.S.C. § 111 (a) and claims priority to U.S. Provisional Patent Application No. 63/618,427 filed on Jan. 8, 2024 in the name of Yihua Bruce Y U, et al., and entitled “Cell Count and Cell Viability Assessment Using Water Proton NMR,” which is hereby incorporated by reference herein in its entirety.

### STATEMENT REGARDING FEDERALLY SPONSORED RESEARCH

This invention was made with United States Government support from the National Institute of Standards and Technology. The Government has certain rights in the invention.

### FIELD

The present invention relates to methods for non-invasive estimates of cell counts and cell viability in cell cultures using solvent nuclear magnetic resonance relaxometry (NMR). The methods can be used to determine the counts of live and dead cells in cell cultures without sample preparation and/or staining.

### DESCRIPTION OF THE RELATED ART

Data on cell counts, including total cell counts and the counts of the viable/live cells, are an indispensable part of many biological experiments as well as biomanufacturing processes. Such data is critically important for cell transfection in biological research, when using cell cultures during the production of biopharmaceuticals, and in drug discovery/design when evaluating the effects of a drug candidate on the viability and proliferation of cells.

Most current methods used for direct cell counting and viable cell estimates are invasive and time consuming. Many use small aliquots taken from the cell culture, which may not be representative of the whole sample. Many require sample preparation to obtain viable cell counts. Prior art methods include incubating the culture sample with a dye that is expected to be excluded from the membranes of viable cells followed by the microscopic examination of one or multiple calibrated microvolume(s) for direct counting of stained and unstained cells to determine concentrations of each. Manual microscopic examination and human counting is still in use alongside instruments that automate the sample preparation, the microscopic examination, and the counting of individual live and dead cells using machine vision or other image classification approaches. Methods based on the Coulter principle, in which the electrical impedance of individual cells flowing between two electrodes is measured, are common alternatives for total cell (particle) counting, but such methods typically require an orthogonal detection technique such as fluorescent dye exclusion to determine the viability of each cell counted. Other techniques measure the bulk properties of cell-containing suspensions (i.e., cultures) such as optical reflectance or capacitance and correlate these findings to cell number concentration.

In practice, prior art methods of cell counting such as the Beckman Coulter Vi-CELL XR specifies its cell count accuracy is ±6% for concentrations of 5×10⁴-1×10⁷ cells/mL. This type of cell counter/analyzer requires removing a sample from a bioreactor, and the sampling process can create an additional source of variability—the technician must be experienced and also careful to draw a representative sample from the bioreactor in order to get a reasonably accurate result from the cell counter; if there are aggregates in the sample, it may be very difficult to achieve an accurate and reproducible result. A 2015 paper (Cadena-Herrera D et al., Biotechnol Rep (Amst), 2015, 7, 9-16) compared viable cell counts using three different methods (e.g., manual, semi-automated, automated), and concluded that the relative standard deviation was 8.06% using the manual hemocytometer method and ≤5.28% using the automated Vi-CELL XR method.

Recently, it has become clear that treatment using autologous cell therapy (CAR-T cells) is in a dire need of accurate cell counts to determine or monitor overall efficacy, safety, treatment response, and assessments of CAR-T cells viability over time. The development and production of these cells involve complex, costly, and resource-demanding procedures, and this makes them extremely valuable components of therapeutic strategies. Therefore, the non-invasive methods of CAR-T cell counting which are non-destructive and permit the use of the actual sample comprising the cells after counting, will significantly reduce costs and healthcare burden while simultaneously supporting continued research and development efforts in the evolving innovative cellular immunotherapy field.

There is clearly a need for a fast and simple technique which can be used to generate reliable cell counts and estimates of the viable cell numbers. Ideally, such a technique should be fast, straightforward, and non-invasive, e.g., does not require addition of any staining dye, does not alter or destroy the sample, and retains the integrity of the sample for further safe usage. With this in mind, a method of using NMR relaxation rates (e.g., the transverse relaxation rate of a solvent NMR signal, or the longitudinal relaxation rate of a solvent NMR signal) as a probe to quantitate the cell counts and viable cell numbers in a cell culture without opening the container and/or without using any staining/labeling reagents is described.

### SUMMARY

The present invention generally relates to a method of using NMR relaxation rates, such as the longitudinal relaxation rate constant R₁ and transverse relaxation rate constant R₂ (e.g., the transverse relaxation rate constant R₂(¹H₂O) of water), to non-invasively estimate cell counts in the cell cultures prepared under similar conditions and to provide the approximate percentage of the viable cells in a cell culture. In some embodiments, the method can be used to monitor the lifecycle of a cell culture via the changes in a cell media. In some embodiments, the method can be used to monitor cell growth in cell cultures providing accurate cell counts on different days of the lifecycle.

In some aspects, a method of estimating a cell count in an aliquot comprising cells is described, said method comprising measuring the relaxation rate of solvent R in the aliquot comprising cells, and comparing the measured R of solvent to a calibration curve to estimate the cell count of the aliquot comprising cells,

wherein the R is selected from the transverse relaxation rate of solvent R₂ or the longitudinal relaxation rate of solvent R₁.

In some other aspects, a method of estimating a percent cell viability in an aliquot comprising cells is described, said method comprising measuring the relaxation rate of solvent R in the aliquot comprising cells, and comparing the measured R of solvent to a calibration curve to estimate the percent cell viability of the aliquot comprising cells,

wherein the R is selected from the transverse relaxation rate of solvent R₂ or the longitudinal relaxation rate of solvent R₁.

In some other aspects, a method of estimating a cell count in an aliquot taken from the growing cell culture is described, said method comprising measuring the relaxation rate of solvent R in the aliquot comprising cells, and comparing the measured R of solvent to a calibration curve to estimate the cell count of the aliquot comprising cells, wherein the R is selected from the transverse relaxation rate of solvent R₂ or the longitudinal relaxation rate of solvent R₁.

Different embodiments, features and advantages of the invention will be more fully apparent from the disclosure below and appended claims.

### BRIEF DESCRIPTION OF THE DRAWINGS

FIG. 1 shows linear dependencies of water proton transverse relaxation rate R₂(¹H₂O) on the total Chinese hamster ovary (CHO) cell counts observed for the same cell harvest (Harvest 1) taken 1 week apart. Data for zero cell count correspond to fresh propagation medium. Fresh Harvest 1, 97% viable cells (open circles), linear fit: slope 0.020±0.002 s⁻¹/(10⁶ cells per mL); <r²>=0.981. One week old Harvest 1, 70% viable cells (solid circles), linear fit: slope 0.028+0.002 s⁻¹/(10⁶ cells per mL); <r²>=0.988. Error bars (±0.002-0.009 s⁻¹) represent the SD of the averages of three consecutive measurements.

FIG. 2 compares linear dependencies of water proton transverse relaxation rate R₂(¹H₂O) on the total CHO cell counts observed for two different fresh cell harvests (Harvest 1 and Harvest 2) cultured 1 month apart. Both harvests show close viable cell counts ˜97%. Harvest 1, 97% viable cells (open circles), linear fit: slope 0.020±0.002 s⁻¹/(10⁶ cells per mL); <r²>=0.981. Harvest 2, 97% viable cells (open triangles), linear fit: slope 0.020±0.0005 s⁻¹/(10⁶ cells per mL); <r²>=0.998. Data for zero cell count correspond to fresh propagation medium. Error bars (+0.002-0.009 s⁻¹) represent the SD of the averages of three consecutive measurements.

FIG. 3 presents linear dependencies of water proton transverse relaxation rate R₂(¹H₂O) on the total CHO cell counts observed for the same cell harvest (Harvest 2) taken 1 month apart. Fresh Harvest 2, 97% viable cells (open triangles), linear fit: slope 0.020±0.0005 s⁻¹/(10⁶ cells per mL); <r²>=0.998; data for zero cell count correspond to fresh propagation medium. One month old Harvest 2, 11% viable cells (solid triangles), linear fit: slope 0.015±0.0005 s⁻¹/(10⁶ cells per mL); <r²>=0.997; data for zero cell count correspond to conditioned medium of initial cell culture containing 6.7×10⁶ cell per mL. Error bars (±0.002-0.009 s⁻¹) represent the SD of the averages of three consecutive measurements.

FIG. 4 shows the increase in water proton transverse relaxation rate R₂(¹H₂O) observed in the cell-free conditioned medium compared to the cell-free fresh propagation medium. PM, is the cell-free fresh propagation medium; Fresh CM, is the cell-free conditioned medium of the fresh Harvest 2 CHO cell culture; 10 d CM, is the cell-free conditioned medium of the 10 days old Harvest 2 CHO cell culture. Harvest 2 CHO cell culture contained 6.1×10⁶ cells per mL, 97% of viable cells in the fresh culture; 85% of viable cells in the 10 days old culture. Error bars (+0.001-0.004 s⁻¹) represent the SD of the averages of three consecutive measurements.

FIG. 5A shows linear dependences of water proton transverse relaxation rate R₂(¹H₂O) on the total CHO cell counts observed during the fed-batch phase of the cell culture growth. Cell viability on each day of the fed-batch (Days 0-3) was >95%. Measurements were performed at three different values of interpulse delay τ of the CPMG pulse sequence. Linear fits for τ=0.5 ms: slope 0.021±0.007 s⁻¹/(10⁶ cells per mL), <r₂>=0.808; for τ=3 ms: slope 0.049±0.024 s⁻¹/(10⁶ cells per mL), <r₂>=0.675; for τ=5 ms: slope 0.065±0.005 s⁻¹/(10⁶ cells per mL), <r₂>=0.986. Error bars (0.002-0.009 s⁻¹) represent the SD of averages of the three consecutive measurements.

FIG. 5B depicts the growth of water proton transverse relaxation rate R₂(¹H₂O) with the increase of total CHO cell counts observed during Days 6-7 after the start of the feeding schedule when cell counts reached ca. 3×10⁶ cells per mL (happened after Day 3). Cell viability on Days 6-7 was >95%. Measurements were performed at three different values of interpulse delay τ of the CPMG pulse sequence: 0.5 ms, 3 ms, and 5 ms. Error bars (0.002-0.009 s⁻¹) represent the SD of averages of the three consecutive measurements.

FIG. 5C shows linear dependences of water proton transverse relaxation rate R₂(¹H₂O) on the total CHO cell counts observed during the feeding schedule after the transfer of the cell culture aliquot to the new flask on Day 7. Fresh feed media mixture was added, so total cell count on Day 8 was 0.8×10⁶ cells per mL. Cell viability on each day of the feeding schedule (Days 8-14) was >95%. Measurements were performed at three different values of interpulse delay τ of the CPMG pulse sequence. Linear fits for τ=0.5 ms: slope 0.016±0.003 s⁻¹/(10⁶ cells per mL), <r₂>=0.923; for τ=3 ms: slope 0.018±0.0005 s⁻¹/(10⁶ cells per mL), <r₂>=0.997; for τ=5 ms: slope 0.019±0.0009 s⁻¹/(10⁶ cells per mL), <r₂>=0.999. Error bars (0.002-0.009 s⁻¹) represent the SD of averages of the three consecutive measurements.

### DETAILED DESCRIPTION, AND PREFERRED EMBODIMENTS THEREOF

The present method generally relates to a technology based on the NMR relaxometry to detect solvent longitudinal and/or transverse relaxation times or rates, e.g., relaxation times and rates of water molecules, to non-invasively estimate total cell counts in a cell culture and provide the percentage of the viable cells in said cell culture. In some embodiments, a method of using NMR relaxation rates (e.g., longitudinal and transverse relaxation rate constants, R₁ and R₂, respectively) of water molecules to non-invasively estimate total cell counts in a cell culture and provide the percentage of the viable cells in said cell culture as well as to monitor the lifecycle of a cell culture via the changes in a cell media. In some embodiments, the method can be used to monitor cell growth in cell cultures providing accurate cell counts on different days of the lifecycle.

As defined herein, a “vial” corresponds to a container, vessel, bottle, syringe, injection pen, or ampoule used to store the product, wherein the vial comprises glass, plastic, ceramic, rubber, elastomeric material, and/or any non-magnetic metal. The vial can have a top including, but not limited to, a screw top, a top that is closed using a cork or plastic stopper, a crimp vial (closed with a rubber stopper and a metal cap), a flip-top or snap cap. The vial can be tubular or have a bottle-like shape with a neck. Other types and shapes of vials used to store products as well as caps are readily understood by the person skilled in the art. The vials can be optically transparent or not optically transparent. There is no need to peel off any label on the vial, whether the label is transparent or not.

As defined herein, a “non-destructive” measurement is defined as a measurement performed without opening the vial or otherwise accessing, harming, or altering the product contained in the vial (for example by withdrawing a portion through a rubber gasket). Alternatively, or in addition to not accessing the contents of a vial, a non-destructive measurement means that no additives or probes or the like (e.g., magnetic particles and/or dyes) are added to the vial prior to the measurement of the longitudinal (T₁) or transverse (T₂) relaxation time of solvent or the longitudinal (R₁) or transverse (R₂) relaxation rate of solvent, e.g., water, in the vial. Non-destructive also means that there is no need to make the vials optically transparent and no need to peel off any labels on the vials.

“Substantially devoid” is defined herein to mean that none of the indicated substance is intentionally added to or present in the product. Alternatively, “substantially devoid” can mean that the amount of the indicated substance in the product is less than about 0.1% (w/w), or less than about 0.05% (w/w), or less than about 0.01% (w/w).

As used herein, “cells” can include, but are not limited to, the cells described hereinafter. In some embodiments, cells are eukaryotic or prokaryotic cells. In some embodiments, cells are mammalian cells (e.g., human cells, canine cells, bovine cells, ovine cells, feline cells, or rodent cells such as rabbit, mouse, or rat cells, such as the Chinese Hamster Ovary (CHO) cells). In some embodiments, cells are insect cells, avian cells, microbial cells (e.g., yeast cells such as Saccharomyces cerevisiae, Kluyveromyces lactis, or Pischia pastoris cells, or bacterial cells such as Escherichia coli, Bacillus subtilis, or Corynebacterium cells), insect cells (e.g., Drosophila cells, or Sf9 or Sf21 cells), plant cells (e.g., algal cells) or cells of any other type. In some embodiments, cells are cultured for producing natural products (e.g., taxols, pigments, fatty acids, biofuels, etc.). In some embodiments, cells are cultured to express recombinant products (e.g., recombinant protein products such as antibodies, hormones, growth factors, or other therapeutic peptides or proteins). In some embodiments, cells are expanded and/or differentiated for therapeutic use such as implantation into a subject (e.g., a human subject) in order to provide or supplement a cellular, tissue, or organ function that is missing or defective in the subject. In some embodiments, cells are from immortalized cell lines including, but not limited to, human cells, e.g., HeLa cells, prostate cancer cells, breast cancer cells, acute myeloid leukemia cells, glioblastoma cells, neuroblastoma cells, bone cancer cells and chronic myelogenous leukemia. In some embodiments, cell lines include primate cell lines, rodent cell lines (e.g., rat or mouse cell lines), canine cell lines, feline cell lines, Zebrafish cell lines, Xenopus cell lines, plant cell lines, or any other cell. In some embodiments, cells are human 293 cells (e.g., 293-T or HEK 293 cells), murine 3T3 cells, Chinese hamster ovary (CHO) cells, CML T1 cells, or Jurkat cells. In some embodiments, cells are primary cells, feeder cells, or stem cells. In some embodiments, cells are isolated from a subject (e.g., a human subject). In some embodiments, cells are primary cells isolated from a tissue or a biopsy sample. In some embodiments, cells are hematopoietic cells. In some embodiments, cells are stem cells, e.g., embryonic stem cells, mesenchymal stem cells, cancer stem cells, etc. In some embodiments, cells are isolated from a tissue or organ (e.g., a human tissue or organ), including but not limited to, solid tissues and organs. In some embodiments, cells can be isolated from placenta, umbilical cord, bone marrow, liver, blood, including cord blood, or any other suitable tissue. In some embodiments, patient-specific cells are isolated from a patient for culture (e.g., for cell expansion and optionally differentiation) and subsequent re-implantation into the same patient or into a different patient. In some embodiments, the cells may be used for allogenic or autogeneic therapy. In some embodiments, the cells may be genetically modified, expanded and reintroduced into a patient for the purpose of providing an immunotherapy (e.g., chimeric antigen receptor (CAR) cells for CAR-therapy (CAR-T), or delivery of CRISPR/Cas modified cells). In some embodiments, a primary cell culture includes epithelial cells (e.g., corneal epithelial cells, mammary epithelial cells, etc.), fibroblasts, myoblasts (e.g., human skeletal myoblasts), keratinocytes, endothelial cells (e.g., microvascular endothelial cells), neural cells, smooth muscle cells, hematopoietic cells, placental cells, or a combination of two or more thereof. In some embodiments, cells are recombinant cells (e.g., hybridoma cells or cells that express one or more recombinant products). In some embodiments, cells are infected with one or more viruses.

In some embodiments, cells are cultured in one of any suitable culture media. Different culture media having different ranges of pH, glucose concentration, growth factors, and other supplements can be used for different cell types or for different applications. In some embodiments, custom cell culture media or commercially available cell culture media such as Dulbecco's Modified Eagle Medium, Minimum Essential Medium, RPMI medium, HA or HAT medium, or other media available from Life Technologies or other commercial sources can be used. In some embodiments, cell culture media include serum (e.g., fetal bovine serum, bovine calf serum, equine serum, porcine serum, or other serum). In some embodiments, cell culture media are serum-free. In some embodiments, cell culture media include human platelet lysate (hPL). In some embodiments, cell culture media include one or more antibiotics (e.g., actinomycin D, ampicillin, carbenicillin, cefotaxime, fosmidomycin, gentamycin, kanamycin, neomycin, penicillin, penicillin streptomycin, polymyxin B, streptomycin, tetracycline, or any other suitable antibiotic or any combination of two or more thereof). In some embodiments, cell culture media include one or more salts (e.g., balanced salts, calcium chloride, sodium chloride, potassium chloride, magnesium chloride, etc.). In some embodiments, cell culture media include sodium bicarbonate. In some embodiments, cell culture media include one or more buffers (e.g., HEPES or other suitable buffer). In some embodiments, one or more supplements are included. Non-limiting examples of supplements include reducing agents (e.g., 2-mercaptocthanol), amino acids, cholesterol supplements, vitamins, transferrin, surfactants (e.g., non-ionic surfactants), CHO supplements, primary cell supplements, yeast solutions, or any combination of two or more thereof. In some embodiments, one or more growth or differentiation factors are added to cell culture media. Growth or differentiation factors (e.g., WNT-family proteins, BMP-family proteins, IGF-family proteins, etc.) can be added individually or in combination, e.g., as a differentiation cocktail comprising different factors that bring about differentiation toward a particular linkage. Growth or differentiation factors and other aspects of a liquid media can be added using automated liquid handlers integrated within the incubators.

“Chimeric antigen receptor” (CAR) T cells are T cells that have been genetically engineered to produce an artificial T-cell receptor. CARs, also known as chimeric immunoreceptors, chimeric T cell receptors or artificial T cell receptors, are receptor proteins that have been engineered to give T cells the ability to target a specific antigen. The receptors are chimeric because they combine both antigen-binding and T-cell activating functions into a single receptor. In more detail, CARs are generally composed of three regions or domains: an ectodomain, a transmembrane domain, and an endodomain.

As used herein, an “aliquot comprising cells” is intended to describe a volume comprising cells having an unknown cell count or an unknown percent cell viability before the user practices the methods described herein to determine same. For example, an aliquot comprising cells can be a cell culture, a sample associated with the evaluation of the effects of a drug candidate on cells, a sample of thawed previously cryopreserved cells, a sample comprising a resuspended cell pellet, or a cell sample aliquoted from the bioreactor.

As used herein, when the “media of the at least one calibration sample and the aliquot comprising cells are substantially the same,” it is understood by the person skilled in the art that the compositional makeup or the milieu of the at least one calibration sample and the aliquot comprising cells are at least 99% identical, or at least 99.5% identical, or more.

As used herein, “substantially the same,” with regards to the cell viability, is intended to be +/−5%, preferably +/−3%, more preferably +/−2%, or even more preferably +/−1%.

As used herein, a “cell preparation” includes, but is not limited to, a cell culture, a sample associated with the evaluation of the effects of a drug candidate on cells, a sample of thawed previously cryopreserved cells, or a sample comprising a resuspended cell pellet.

As used herein, “substantially identical conditions” of a cell harvest or culture include at least one of cell type, time, temperature, CO₂%, agitation, and/or propagation medium.

As used herein, “relaxivity” or r₂, is the slope of the R₂(¹H₂O) vs. cell count. Relaxivity correlates with cell viability and can be used as a quality attribute of a cell preparation/sample.

Advances in the instrumentation for compact low-field nuclear magnetic resonance (NMR) spectroscopy and magnetic resonance imaging (MRI) resulted in the opportunities to develop novel nondestructive analytical techniques for the pharmaceutical industry (Michal, C.A. 2020. J. Magn. Reson. 319:106800). Low-field benchtop NMR instruments allow the user to measure nuclear spin relaxation times T₁ and T₂ with high accuracy. Currently, almost all of these benchtop instruments have a permanent magnet with the wide bore from about 10 mm to about 45 mm, or even larger, which provides for the non-invasive measurement of pharmaceutical products in their original containers including, but not limited to, vials or syringes, of various sizes.

Water proton NMR technology (wNMR) analyzes the dynamics of water signal, wherein the water acts as a probe for cells, analytes, particles, etc. dissolved and/or suspended in it. As a reporter, water has many advantages. First, its concentration far surpasses that of any analyte dissolved in it, by 10³-10⁶ fold in most cases. This makes the ¹H₂O signal easily detectable by NMR instruments, including low-field NMR instruments, with high signal-to-noise ratio. Further, the water proton NMR signal is very sensitive to various changes in solute content, its organization/association, and its degradation because water is “endogenous” to all biomanufacturing processes and all pharmaceutical products, including cell cultures. The high concentration of “endogenous” water makes it possible for wNMR to be non-invasive and contact-free in situ for cell culture analysis without the addition of any reagents and/or staining dyes. Although only examples of products formulated with water as the solvent are presented in this patent application, the extension of NMR to products formulated with other solvents (e.g., ethanol) is contemplated herein.

wNMR is a useful characterization tool for the pharmaceutical industry, thereby allowing the manufacturing and research industry to explore the status of a cell culture for further use or corrective actions. For example, in some embodiments, wNMR may be used to determine total cell counts in a culture. In some embodiments, wNMR may be used to determine the percent of the viable cells in a culture. In some embodiments, wNMR might be used for indirect monitoring of the cells' lifecycle based on the changes detected in cell media.

The method described herein is a reliable and simple method to assess the total cell counts in a culture, and to determine the percentage of the viable cells in a culture. The method enables the non-invasive analysis of a cell culture sample aliquoted or otherwise taken from bioreactor, and also allows for the monitoring of changes in the said bioreactor during the cells' lifecycle or biomanufacturing. In some embodiments, the method is quantitative and comprises determining the nuclear spin relaxation rate constant, R₁ and/or R₂, of solvent as an indicator. In some embodiments, the method is quantitative and comprises determining the nuclear spin relaxation rate constant, R₁(¹H₂O) and/or R₂(¹H₂O), of water as an indicator. There exists a quantitative variation of the nuclear spin relaxation rate constant, R₁ and/or R₂, of solvent, e.g., water, that is dependent on the total cell count in a sample, thus permitting the determination of the percentage of viable cells in a sample. Also, the said nuclear spin relaxation rates of solvent, e.g., water, are sensitive to the concentration of metabolites excreted by cells in a culture media which reflects the growth and aging of a cell culture. Moreover, the methods described herein have a low percentage of error and is easy to use.

The present inventors have surprisingly discovered that solvent NMR can be used to estimate the total cell count in a cell culture, the percentage of viable cells, and monitor cell growth and aging during the cell lifecycle with high reproducibility and small variability compared to the prior art cell counting techniques. In addition to being non-invasive, additional advantages of solvent NMR includes low-cost instrumentation (e.g., a desktop or handheld NMR), simple and rapid data acquisition and analysis (e.g., within <1-2 min), and minimal technical expertise requirement. It should be appreciated that the measurements can occur destructively as well, whereby the vial is opened, if needed. Further, the method described herein can utilize high field NMR, if needed.

Broadly, in a first aspect, a method of estimating cell counts using relaxation rates of solvent R is described.

In some embodiments of the first aspect, a method of estimating a cell count in an aliquot comprising cells is described, said method comprising measuring the relaxation rate of solvent R in the aliquot comprising cells, and comparing the measured R of solvent to a calibration curve to estimate the cell count of the aliquot comprising cells,

wherein the R is selected from the transverse relaxation rate of solvent R₂ or the longitudinal relaxation rate of solvent R₁.

In some embodiments of the first aspect, a method of estimating a cell count in an aliquot taken from the growing cell culture is described, said method comprising measuring the relaxation rate of solvent R in the aliquot comprising cells, and comparing the measured R of solvent to a calibration curve to estimate the cell count of the aliquot comprising cells,

wherein the R is selected from the transverse relaxation rate of solvent R₂ or the longitudinal relaxation rate of solvent R₁.

In some embodiments, the solvent comprises water. In some embodiments, the aliquot comprising cells is a cell culture, a sample associated with the evaluation of the effects of a drug candidate on cells, a sample of thawed previously cryopreserved cells, or a sample comprising a resuspended cell pellet. In some embodiments, the aliquot is contained in a vial. In some embodiments, the aliquot passes through a conduit communicatively connected to a bioreactor, wherein the conduit passes through an NMR magnet and NMR probe cavity, and wherein the process is in-line and contact-free and is monitored in real-time by wNMR. In some embodiments, the relaxation rate of solvent R is measured without adding any additives to the vial containing the aliquot. In some embodiments, the additives are selected from staining dyes and magnetic particles. In some embodiments, the relaxation rate of solvent R is measured using nuclear magnetic resonance (NMR) or magnetic resonance imaging (MRI). In some embodiments, the calibration curve is prepared by plotting relaxation rate R versus a known cell count of at least one calibration sample. In some embodiments, media of the at least one calibration sample and the aliquot comprising cells are substantially the same. In some embodiments, the at least one calibration sample and the aliquot comprising cells were cultured under substantially identical conditions. In some embodiments, the measurement of the relaxation rate of solvent R is performed at a same temperature used to generate the calibration curve. In some embodiments, the measurement of the relaxation rate of solvent R is performed at a same magnetic field strength used to generate the calibration curve. In some embodiments, the method is non-destructive. In some embodiments, following measurement of the relaxation rate of solvent R, the aliquot comprising the cells is not altered and can continue to be used. In some embodiments, R is the transverse relaxation rate of solvent R₂. In some embodiments, R is the longitudinal relaxation rate of solvent R₁. In some embodiments, the R₂ measurement variability is less than 1%, which is much improved over the methods/techniques of the prior art.

In practice, to use relaxation rates R (or times T) to estimate cell counts, the cell count of at least one calibration sample is determined using automated cell counter/analyzers known in the art. These include fairly precise bench-top instruments (e.g., Beckman Coulter Vi-CELL) as well as in situ process measurement of total cell density & viable cell density in large-scale biorcactors (e.g., Hamilton VCD and TCD sensors). Next, the relaxation rate R (or time T) of the at least one calibration sample is measured, a calibration curve of cell count versus relaxation rate R (or time T) is prepared, and a best-fit regression line is obtained. Thereafter, the relaxation rates R (or times T) of the aliquot comprising cells (e.g., having an unknown cell count) from harvests cultured under substantially identical conditions as the at least one calibration sample can be measured and the calibration curve used to estimate the cell count. In some embodiments, so long as the aliquot comprising cells (e.g., having an unknown cell count) is obtained from a harvest cultured under substantially identical conditions as the at least one calibration sample, whether one week after, one month after, or one year after, no additional validation by cell counters/analyzers is needed.

In a second aspect, a method of estimating percent cell viability using relaxation rates of solvent R is described.

In some embodiments of the second aspect, a method of estimating a percent cell viability in an aliquot comprising cells is described, said method comprising measuring the relaxation rate of solvent R in the aliquot comprising cells, and comparing the measured R of solvent to a calibration curve to estimate the percent cell viability of the aliquot comprising cells,

wherein the R is selected from the transverse relaxation rate of solvent R₂ or the longitudinal relaxation rate of solvent R₁.

In some embodiments, the solvent comprises water. In some embodiments, the aliquot comprising cells is a cell culture, a sample associated with the evaluation of the effects of a drug candidate on cells, a sample of thawed previously cryopreserved cells, or a sample comprising a resuspended cell pellet. In some embodiments, the aliquot is contained in a vial. In some embodiments, the aliquot passes through a conduit communicatively connected to a bioreactor, wherein the conduit passes through an NMR magnet and NMR probe cavity, and wherein the process is in-line and contact-free and is monitored in real-time by wNMR. In some embodiments, the relaxation rate of solvent R is measured without adding any additives to the vial containing the aliquot. In some embodiments, the additives are selected from staining dyes and magnetic particles. In some embodiments, the relaxation rate of solvent R is measured using nuclear magnetic resonance (NMR) or magnetic resonance imaging (MRI). In some embodiments, the calibration curve is prepared by plotting relaxation rate R versus a known percent cell viability of at least one calibration sample. In some embodiments, media of the at least one calibration sample and the aliquot comprising cells are substantially the same. In some embodiments, the at least one calibration sample and the aliquot comprising cells were cultured under substantially identical conditions. In some embodiments, the measurement of the relaxation rate of solvent R is performed at a same temperature used to generate the calibration curve. In some embodiments, the measurement of the relaxation rate of solvent R is performed at a same magnetic field strength used to generate the calibration curve. In some embodiments, the method is non-destructive. In some embodiments, following measurement of the relaxation rate of solvent R, the aliquot comprising the cells can be used. In some embodiments, R is the transverse relaxation rate of solvent R₂. In some embodiments, R is the longitudinal relaxation rate of solvent Ry. In some embodiments, the R₂ measurement variability is less than 1%, which is much improved over the methods/techniques of the prior art.

In practice, to use relaxation rates R (or times T) to estimate percent cell viability, the percent cell viability of at least one calibration sample is determined using automated cell counter/analyzers known in the art such as precise bench-top instruments (e.g., Beckman Coulter Vi-CELL) as well as in situ process measurement of total cell density & viable cell density in large-scale bioreactors (e.g., Hamilton VCD and TCD sensors). Next, the relaxation rate R (or time T) of the at least one calibration sample is measured, a calibration curve of percent cell viability versus relaxation rate R (or time T) is prepared, and a best-fit regression line is obtained. Thereafter, the relaxation rates R (or times T) of the aliquot comprising cells (e.g., having an unknown percent cell viability) from harvests cultured under substantially identical conditions as the at least one calibration sample can be measured and the calibration curve used to estimate the percent cell viability. In some embodiments, so long as the aliquot comprising cells (e.g., having an unknown percent cell viability) is obtained from a harvest cultured under substantially identical conditions as the at least one calibration sample, whether one week after, one month after, or one year after, no additional validation by cell counters/analyzers is needed. In some embodiments, a cell culture can be artificially aged to obtain at least one calibration sample having a known percent cell viability (e.g., depending on the aging time and temperature) to create a viability calibration curve, which can be used to analyze an aliquot comprising cells (e.g., having an unknown percent cell viability) from harvests cultured under substantially identical conditions and aged for the same length of time as the at least one calibration sample can be measured and the calibration curve used to estimate the percent cell viability. In some embodiments, so long as the aliquot comprising cells (e.g., having an unknown percent cell viability) is obtained from a harvest cultured under substantially identical conditions for the same length of time as the at least one calibration sample, no additional validation by cell counters/analyzers is needed.

It should be appreciated that the methods described herein can be based on the solvent proton transverse relaxation time T₂ (instead of the rate R₂) or the solvent proton longitudinal relaxation time T₁ (instead of the rate R₁), as readily determined by the person skilled in the art.

Advantageously, the methods of the first or second aspect described herein are non-invasive, as opposed to other currently used cell counting methods, and do not require any sample preparations, aliquoting or staining. There is low measurement variability because no sample preparation is needed. For cell therapy products (e.g., CAR-T cells), where the cells are produced at small scale for therapy (vs. CHO cells used in biomanufacturing which are produced at large scale), wNMR can be used for quality control without destroying the sample, i.e., cells analyzed by wNMR can still be used for treating patients.

In some embodiments of the first or second aspects described herein, the aliquot comprising cells can be obtained by withdrawing a sample from a bioreactor comprising the cells, as understood by the person skilled in the art. In some embodiments, the aliquot comprising cells can be a resuspended cell pellet. In some other embodiments, in situ real-time in-line detection using NMR relaxation rates can be used. For example, as described in U.S. Pat. No. 11,543,371 in the name of Yihua (Bruce) Yu et al. and entitled “In Situ, Real-Time In-Line Detection of Filling Errors in Pharmaceutical Product Manufacturing Using Water Proton NMR,” which is hereby incorporated herein in its entirety, a custom-made flow NMR instrument was described to analyze the changes of NMR relaxation rates due to changes of flow rate or concentration under flow. There is no drawing of any sample out of the flow loop and there is no physical contact between the NMR instrument and the sample, e.g., cell culture, which stays inside a closed loop that passes through the NMR magnet/probe. There was no need for bypass system and no stop-flow cell, although they can be accommodated. The results demonstrated in U.S. Pat. No. 11,543,371 show that flow-wNMR, wherein a looped conduit passes through an NMR magnet, wherein an aliquot comprising cells withdrawn or removed from a bioreactor is returned to the bioreactor after passing through the conduit passing through the magnet, could be used as a contact-free in-line process analytical technology (PAT) that can estimate cell counts or percent cell viability in real time without withdrawing, or directly contacting, any sample from the bioreactor comprising a cell harvest.

Accordingly, in some embodiments of the first aspect, a method of estimating a cell count in an aliquot comprising cells is described, said method comprising:
flowing the aliquot comprising cells through a conduit, wherein the conduit is communicatively connected to a bioreactor and is arranged to flow through a magnet and probe cavity of a nuclear magnetic resonance (NMR) spectrometer prior to returning to the bioreactor via the conduit; measuring the relaxation rate of solvent R in the aliquot comprising cells, and comparing the measured R of solvent to a calibration curve to estimate the cell count of the aliquot comprising cells,
wherein the R is selected from the transverse relaxation rate of solvent R₂ or the longitudinal relaxation rate of solvent R₁.

Accordingly, in some embodiments of the second aspect, a method of estimating a percent cell viability in an aliquot comprising cells is described, said method comprising:
flowing the aliquot comprising cells through a conduit, wherein the conduit is communicatively connected to a bioreactor and is arranged to flow through a magnet and the probe cavity of a nuclear magnetic resonance (NMR) spectrometer prior to returning to the bioreactor via the conduit;
measuring the relaxation rate of solvent R in the aliquot comprising cells; and
comparing the measured R of solvent to a calibration curve to estimate the percent cell viability of the aliquot comprising cells, wherein the R is selected from the transverse relaxation rate of solvent R₂ or the longitudinal relaxation rate of solvent R₁.

In some embodiments of the first or second aspect described herein, for a given cell type and cell culture medium, relaxivity (r₂) might serve as a critical quality attribute (CQA) of a cell population as it relates to cell viability. In some embodiments, a cell sample can be characterized by two wNMR parameters: R₂(¹H₂O), which correlates with cell count (number); and r₂, which correlates with cell viability. The supplier of the cell sample should instruct the user on how to measure R₂(¹H₂O) and r₂ for quality verification. In some embodiments, such instructions should include NMR analyzer make/model, pulse sequence, temperature, container type/dimension (e.g., glass vials with an outer diameter 20 mm), and other relevant measurement parameters such as the interpulse delay τ. Additionally, for r₂ measurements, instructions should be provided by the cell supplier on dilution procedure (mixing, shaking, etc.), dilution medium and containers to hold diluted samples. The supplier should measure both R₂(¹H₂O) and r₂ for their product/samples and label their product/samples with such parameters and instructions for how to measure them for the downstream user as quality attributes of a cell preparation/sample. It should be appreciated by the skilled artisan that if you are the supplier, you must determine this information and provide same to the downstream user.

Accordingly, in a third aspect, a method of determining a relaxivity (r₂) value of a cell preparation is described, said method comprising:
(a) measuring the relaxation rate of solvent R of an aliquot of the cell preparation, wherein the cell preparation has a known cell viability and known cell count per unit volume;
(b) optionally, repeating step (a) more than once by varying the known cell count per unit volume of the cell preparation while maintaining the known cell viability;
(c) plotting a graph of R versus cell count, and calculating the slope of a best-fit regression line to determine the relaxivity,
   wherein the R is selected from the transverse relaxation rate of solvent R₂ or the longitudinal relaxation rate of solvent R₁.

In some embodiments, the solvent comprises water. In some embodiments, the aliquot comprising cells is a cell culture, a sample associated with the evaluation of the effects of a drug candidate on cells, a sample of thawed previously cryopreserved cells, or a sample comprising a resuspended cell pellet. In some embodiments, step (b) is performed one time, two times, three times, four times, or more, in addition to step (a), using a range of known cell counts per unit volume to obtain enough R values establish a multi-point line. To determine the relaxivity (r₂) value of a cell preparation, the cell viability of each of the aliquots in step (a) and (b) are substantially the same. In some embodiments, the aliquot is contained in a vial. In some embodiments, the aliquot passes through a conduit communicatively connected to a bioreactor, wherein the conduit passes through an NMR magnet and NMR probe cavity, and wherein the process is in-line and contact-free and is monitored in real-time by wNMR. In some embodiments, the relaxation rate of solvent R is measured without adding any additives to the vial containing the aliquot. In some embodiments, the additives are selected from staining dyes and magnetic particles. In some embodiments, the relaxation rate of solvent R is measured using nuclear magnetic resonance (NMR) or magnetic resonance imaging (MRI). In some embodiments, the measurement of the relaxation rate of solvent R is performed at a same temperature regardless of the cell count. In some embodiments, the measurement of the relaxation rate of solvent R is performed at a same magnetic field strength regardless of the cell count. In some embodiments, the method is non-destructive. In some embodiments, following measurement of the relaxation rate of solvent R, the aliquot comprising the cells can be used. In some embodiments, R is the transverse relaxation rate of solvent R₂. In some embodiments, R is the longitudinal relaxation rate of solvent R₁. In some embodiments, the R₂ measurement variability is less than 1%, which is much improved over the methods/techniques of the prior art.

In some embodiments, the cell count of the cell preparation can be determined using automated cell counter/analyzers known in the art. These include fairly precise bench-top instruments (e.g., Beckman Coulter Vi-CELL) as well as in situ process measurement of total cell density & viable cell density in large-scale bioreactors (e.g., Hamilton VCD and TCD sensors). In some embodiments, the cell count of the cell preparation can be determined using the method of the first aspect, as described herein.

In sum, NMR relaxation rates (or times) can be used to estimate the total cell count in a cell culture, the percentage of the viable cells, the relaxivity (r₂), and also can be used to monitor cell growth and aging during the cells' lifecycle with higher reproducibility and smaller variability compared to the prior art cell counting techniques. Moreover, the combined use of R₂ and r₂ can be used as quality attributes of a cell preparation/sample. NMR relaxation rates (or times) is fast, straightforward, and non-invasive, e.g., does not require addition of any staining dye, does not alter or destroy the sample, and retains its integrity for further safe usage. The R₂ measurement variability is less than 1%, which is much improved over the methods/techniques of the prior art. Moreover, the simplicity of the methods described herein make them well suited for automation.

The present subject matter may be a system, a method, and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The features and advantages of the present invention will be understood more readily by reference to the examples discussed below, which are provided by way of illustration and are not intended to be limiting of the present invention.

### EXAMPLES

### Example 1. Cell Culture and Cell Counts Using Conventional Techniques

Three cell cultures were grown and examined by both the wNMR technique described herein and a prior art cell counting technique using a commercial instrument performing automated sample dilution and mixing with trypan blue solution, automated microscopy of sample under stop-flow, and automated image analysis. All automated image analysis was performed with the same analysis settings using either 50 or 100 images per sample. All cultures were of the same clone of CHO K-1 subtype cells adapted to suspension (non-adherent) cell culture in a chemically defined growth medium. All cultures were grown by incubation in a commercial growth medium of proprietary formulation under conditions of 37° C., 5% carbon dioxide (CO₂) and 80% relative humidity (RH) atmosphere, and agitation by orbital shaking on a 25 mm orbit at 135 rotations per minute (RPM). Passages represent subcultures of prior cultures that have reached exponential growth phase, and passages are numbered from the original research cell bank of the clone used. Culture A was examined in passage 9 after approximately 7 days from inoculation. Culture A was also examined in passage 10 after approximately 3 days from inoculation and after approximately 10 days from inoculation. Culture B and Culture C were revived in passage 10 from a cryopreserved stock of Culture A at passage 6. Culture B was examined in passage 11 after approximately 6 days from inoculation. Culture C was examined in passage 11 after approximately 21 days from inoculation. Growth medium was examined by wNMR as provided by the manufacturer. Culture B in passage 10 and in passage 11 was used as the source of cell-free conditioned culture medium at approximately 6 days and approximately 10 days from inoculation, respectively. Cell-free conditioned culture medium samples were prepared by sampling a volume of the respective culture passage, sedimenting cells by brief centrifugation at 200×g, and passing the resulting supernatant through a sterile 0.2 micron pore diameter syringe-driven membrane filter.

### Example 2. Cell Counts and Cells Viability Monitored by wNMR in the Same Harvest

The sensitivity of R₂(¹H₂O) to total cell counts and the capability of wNMR to monitor cell viability in the same harvest of CHO cells during the life cycle was explored. Fresh Harvest 1 of CHO cells described in the Example 1 containing 6.2×10⁶ cells per mL with 97% viable cells (determined using Beckman Coulter Vi-CELL automated cell counter/analyzer) was used to study the dependence of R₂(¹H₂O) on total cell count in the mature cell culture. The aliquots of the mature cell culture of the fresh Harvest 1 were diluted using fresh propagation medium (4-fold dilution) as well as concentrated by gentle centrifugation at 250 rpm followed by aspiration of about one quarter of the supernatant and resuspension of the cell pellet. This results in a series of samples containing 0, 1.7×10⁶, 6.2×10⁶, and 8.3×10⁶ cells per mL. Cell counts performed in accordance with the protocols described in Example 1 using Beckman Coulter Vi-CELL automated cell counter/analyzer confirmed that the three latter samples contained 97% viable cells.

Two mL aliquots of each sample of the above said series with different cell counts were added to sterile ISO 2R glass vials and capped with sterile rubber septa. All sample preparations were performed under sterile conditions in a biosafety cabinet.

Water proton relaxation of each sample was analyzed at 0.56 T (23.8 MHz 1H resonance frequency, Oxford Instruments MQC+ equipped with a PRO 1193 probe) non-invasively, in the ISO 2R glass vials without opening, sampling and/or adding anything to the cell cultures. Water proton transverse relaxation time T₂(¹H₂O) was measured using Carr-Purcell-Meiboom-Gill (CPMG) pulse sequence. However, it would be evident for the person ordinary skilled in the art that there are other methods to determine T₂(¹H₂O), and the CPMG pulse sequence used herein is not intended to limit the determination of T₂(¹H₂O).

Water proton transverse relaxation time T₂(¹H₂O) measured using CPMG pulse sequence was extracted by fitting experimental spin-echo decay data to Formula (1):

$$I ( t ) = {I}_{0} \times exp ( - t / {T}_{2} {(}^{1} {H}_{2} O ) ) \qquad ( 1 )$$

where I(t) is the water proton echo signal intensity at time t, I₀ is the initial water proton echo signal at t=0, and t is the echo delay time. Measurement parameters included relaxation delay of 12 sec, interpulse delay of 0.5 millisecond (ms), and 15,000 echoes were collected with 4 accumulated transients. The data collection time is less than 2 min. The resulting water proton relaxation time T₂(¹H₂O) values were converted to the water proton relaxation rate R₂(¹H₂O) using Formula (2):

$${R}_{2} {(}^{1} {H}_{2} O ) = 1 / {T}_{2} {(}^{1} {H}_{2} O ) \qquad ( 2 )$$

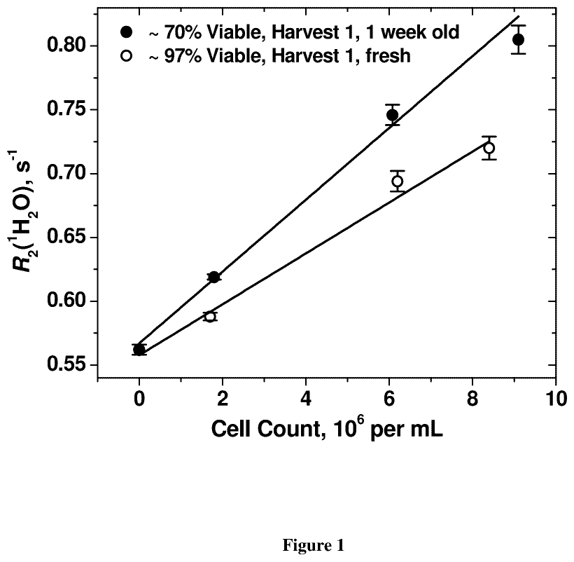

The dependence of water proton transverse relaxation rate R₂(¹H₂O) on the total CHO cell counts for the fresh Harvest 1 CHO cell culture (97% of viable cells) is shown in FIG. 1 (open circles). Excellent linear fit (<r²>=0.981) with the slope to 0.020 s⁻¹/(10⁶ cells per mL) allows for the establishment of a reliable correlation between the observed R₂(¹H₂O) values and the total cell counts in a given sample. The present linear dependence could be advantageously used to reliably determine the total cell counts with a coefficient of variation (CV) of 0.97%. Prior art techniques based on automated microscopy and image analysis have been demonstrated to have total cell count CVs of approximately 3.0%. Counts with CV≤20.0% is a metric used by some in the field to determine the lower limit of quantitation of a method for use with dilute cell suspensions.

The sensitivity of R₂(¹H₂O) to the presence of dead cells in a cell culture with the same total CHO cell counts in the same harvest during its life cycle was explored. One week old Harvest 1 of CHO cells containing a total of 6.1×10⁶ cells per mL with 70% of viable cells was used to study the dependence of R₂(¹H₂O) on total cell count in the mature cell culture. To this end, the aliquots of 1 week old Harvest 1 were also diluted with propagation medium or concentrated in a similar way as described above for the fresh Harvest 1 CHO cell culture. The resulting series included samples containing 0, 1.8×10⁶, 6.1×10⁶, and 9.1×10⁶ cells per mL. Cell counts performed in accordance with the protocols described in Example I confirmed that the three latter samples contained 70% viable cells.

Similar to above, ISO 2R sterile glass vials with sterile rubber septa containing 2 mL aliquots of each sample in a series were also prepared under sterile conditions in the biosafety cabinet. All water proton transverse relaxation experiments with the 1 week old Harvest 1 cell culture samples were performed as described above for the fresh Harvest 1 culture. The same CPMG parameters and data processing procedures used for the fresh Harvest 1 were also employed for the 1 week old Harvest 1 culture.

The dependence of the water proton transverse relaxation rate R₂(¹H₂O) on the total CHO cell counts for the 1-week-old Harvest 1 CHO cell culture (70% of viable cells) is shown in FIG. 1 (solid circles). Similar to the fresh Harvest 1, excellent linear fit (<r²>=0.988) with the slope 0.028 s⁻¹/(10⁶ cells per mL) makes it possible to reliably determine total cell counts from the observed R₂(¹H₂O) values in a given sample with lower counts of viable cells, i.e., a sample containing a mixture of viable and dead cells. The present linear dependence could be advantageously used to reliably determine the total cell counts with a CV of 0.92%. The slope of the R₂(¹H₂O) vs. cell count linear dependence may be termed relaxivity as it describes the ability of cells to facilitate water proton relaxation. Relaxivity depends on cell types, cell culture media and cell viability. For a given cell type and cell culture medium, relaxivity might serve as a critical quality attribute (CQA) of a cell population as it relates to cells viability. Conceivably, a cell sample could be characterized by two wNMR parameters, R₂(¹H₂O), which correlates with cell count (number) and r₂, which correlates with cell viability. The supplier of the cell sample should instruct the user on how to measure R₂(¹H₂O) and r₂ for quality verification. In some embodiments, such instructions should include NMR analyzer make/model, pulse sequence, temperature, container type/dimension (e.g., glass vials with an outer diameter of 20 mm), and other relevant measurement parameters such as the interpulse delay τ. Additionally, for r₂ measurements, instructions should be provided by the cell supplier on dilution procedure (mixing, shaking, etc.), dilution medium and containers to hold diluted samples.

Notably, comparison of the R₂(¹H₂O) versus cell count dependencies for the fresh and 1 week old Harvest 1 cultures in FIG. 1 points to a significant increase of the R₂(¹H₂O) in the samples with the same number of total cells but lower viable cell numbers. Of note, for almost identical total cell counts in the cultures containing 70% and 97% viable cells (6.1×10⁶ vs. 6.2×10⁶ cells per mL, respectively), the difference on the respective R₂(¹H₂O) amounts to 0.052 s⁻¹. Considering 27% difference in the viable cell counts in two compared cultures, this is equivalent to about 0.002 s⁻¹ per each 1% change in the viable cell numbers. Hence, since the R₂(¹H₂O) measurement error is within a range from 0.001 s⁻¹ to 0.002 s⁻¹, this suggests that water proton NMR can provide reliable estimates of cell viability with accuracy from about 3% to about 5%.

### Example 3. Reproducibility of wNMR Measurements in Different Harvests

To explore the reproducibility of the R₂(¹H₂O) measurements of different harvests, the results of the fresh Harvest 1 of CHO cells shown in FIG. 1 (open circles) were compared to the fresh Harvest 2 prepared 1 month later (Example 1). In close similarity with the fresh Harvest 1, the fresh Harvest 2 contained 6.1×10⁶ cells per mL (cf. 6.2×10⁶ cells per mL for the fresh Harvest 1) with approximately identical cell viability (97%, per Example 1). For comparison purposes, an aliquot of the fresh Harvest 2 was diluted using fresh propagation medium (4-fold dilution) which resulted in a series of samples containing 0, 1.1×10⁶, and 6.1×10⁶ cells per mL. Cell counts performed in accordance with the protocols described in Example 1 confirmed that the two latter samples contained approximately 97% viable cells.

Similar to the above description of the Example 2, ISO 2R sterile glass vials with sterile rubber septa containing 2 mL aliquots of each sample of the fresh Harvest 2 in the above series were also prepared under sterile conditions in the biosafety cabinet. All water proton transverse relaxation experiments with the fresh Harvest 2 cell culture samples were performed as described above in the Example 2. Parameters of water proton relaxation were measured using the same CPMG approach as disclosed in the Example 2 herein. Data processing was also the same as in the Example 2 hereinabove.

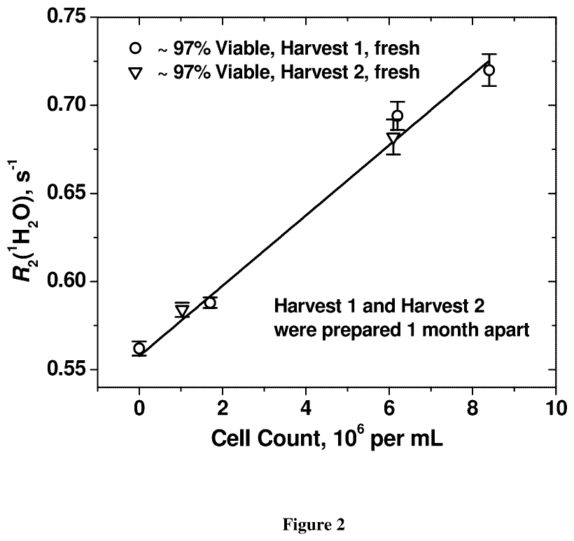

FIG. 2 compares the dependencies of R₂(¹H₂O) vs. total cell counts for the fresh Harvest 1 (open circles) and the fresh Harvest 2 (open triangles) at approximately identical cell viability (97%). Linear dependencies for both harvests demonstrate identical slopes, 0.020 s⁻¹/(10⁶ cells per mL), and excellent fit quality, <r²>>0.98. As it has been mentioned in Example 2, this suggests that for both harvests, these linear dependencies could be advantageously used to reliably determine the total cell counts with the CV 0.97%.

It should be pointed out that both cell cultures of the fresh Harvest 1 and the fresh Harvest 2 were grown under similar conditions and both show almost identical cell counts (6.2×10⁶ and 6.1×10⁶ cells per mL, respectively) with approximately identical fraction of the viable cells (about 97%). Hence, it should be appreciated by the person skilled in the art that wNMR could be advantageously used to determine total cell counts for the harvests cultured under similar conditions in the same propagation medium.

### Example 4. Aged Cell Cultures Explored by wNMR

To delve deeper into the contribution of the growing count of dead cells to the increase of the R₂(¹H₂O) values, the fresh Harvest 2 culture was allowed to age for 1 month. The resulting 1 month old Harvest 2 culture contained 6.7×10⁶ cells per mL with approximately 11% of the cells being viable (i.e., 89% of the cells in the sample are dead). In order to compare with wNMR data obtained for the fresh Harvest 2 (Example 3), a series of the 1 month old Harvest 2 culture samples containing different cell counts were prepared. In this case, an aliquot of the 1 month old Harvest 2 culture was diluted (4-fold dilution) using the conditioned medium of this culture (1 m CM, see Example 1 for preparation procedure). To obtain larger cell counts, an aliquot of the 1 month old Harvest 2 culture was concentrated by gentle centrifugation at 250 rpm followed by aspiration of about one quarter of the supernatant and resuspension of the cell pellet. This allowed for the obtainment of a sample series containing 0, 1.1×10⁶, 6.7×10⁶, and 9.6×10⁶ cells per mL. Note that in this series, the zero cell count sample is a conditioned medium (1 m CM). Cell counts performed in accordance with the protocols described in the Example 1 confirmed that three latter samples contained only 11% of the viable cells.

Same as in the Examples 2 and 3 hereinabove, the sample series were prepared in the ISO 2R sterile glass vials with sterile rubber septa containing 2 mL aliquots of each sample of the 1 month old Harvest 2 from the above series. All procedures were performed under sterile conditions in the biosafety cabinet. All water proton transverse relaxation rate measurements of the 1 month old Harvest 2 samples were performed as described above in the Examples 2 and 3. Same CPMG pulse sequence parameters and data processing protocols were used as disclosed in the Example 2 herein.

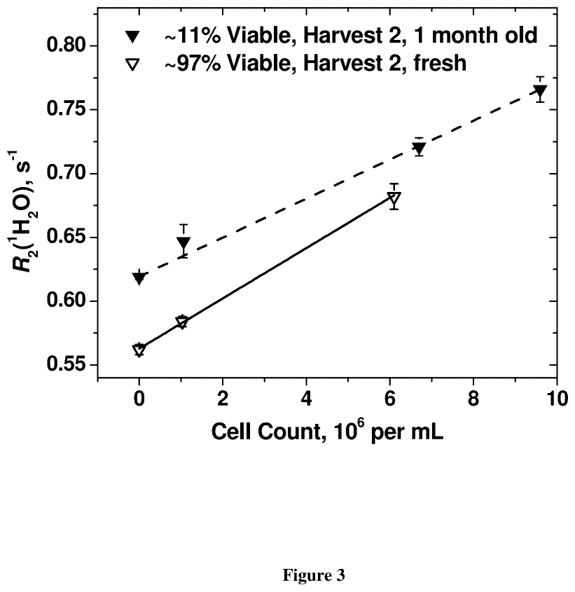

FIG. 3 compares linear dependencies of R₂(¹H₂O) vs. total cell counts for the fresh Harvest 2 (open triangles) with 97% of the viable cells and the 1 month old Harvest 2 (solid triangles) with only 11% of the viable cells. Both linear dependencies show excellent fit quality, <r²>>0.99, however, the slopes demonstrate certain differences, 0.020 vs. 0.015 s⁻¹/(10⁶ cells per mL) for the fresh and 1 month old Harvest 2, respectively. The present linear dependence could be advantageously used to reliably determine the total cell counts with the CV 1.4%.

Note that contrary to Example 2, in this case, it is difficult to provide the approximate accuracy of cell viability estimates from the comparison of two linear dependencies shown in FIG. 3. This is because the sample series of the fresh Harvest 2 and the 1 month old Harvest 2 were prepared using different media. As a reminder, the series of the fresh Harvest 2 samples was prepared using fresh propagation media, whereas the series of the 1 month old Harvest 2 samples was prepared using 1 month old conditioned media (1 m CM). Nevertheless, the findings of this Example 4 unambiguously point to the capability of wNMR to reliably distinguish between fresh and aged cell cultures from the same harvest.

### Example 5. Cell Media Analysis by wNMR

It is worth pointing out that comparison of the slopes of linear dependencies for aged cell cultures vs. fresh ones reveals different trends for the Harvest 1 (FIG. 1) and the Harvest 2 (FIG. 3). Indeed, the slope for the aged culture of the Harvest 1 is larger compared to the fresh one (0.028 vs. 0.020 s⁻¹/(10⁶ cells per mL)), while the slope for the aged culture of the Harvest 2 is smaller compared to the fresh one (0.015 vs. 0.020 s⁻¹/(10⁶ cells per mL)). It might be suggested that such difference is due to the fact that the sample series of the aged culture of Harvest 2 was prepared using the 1 month old conditioned medium (1 m CM, see Example 1 for preparation procedure), while the fresh culture sample series was prepared using fresh propagation medium. Hence, it would appear reasonable that the observed difference is a result of the increasingly greater presence of cell metabolites and debris accumulated in the medium during cell growth and aging.

To explore this assumption water proton relaxation was analyzed in fresh propagation medium, conditioned medium of the fresh cell culture, and conditioned medium of the 10 days old cell culture. Briefly, all media were cell-free, wherein in both conditioned media cells were removed by gentle centrifugation followed by filtration through 0.22 μm filter (see Example 1 for details). Same as in the Examples 2-4, ISO 2R sterile glass vials with sterile rubber septa containing 2 mL aliquots of each sample of the above listed media were prepared under sterile conditions in the biosafety cabinet. All water proton transverse relaxation rate measurements on the media sample were performed as described above in Examples 2-4. The same CPMG pulse sequence parameters and data processing protocols were used as disclosed in the Examples 2-4 herein.

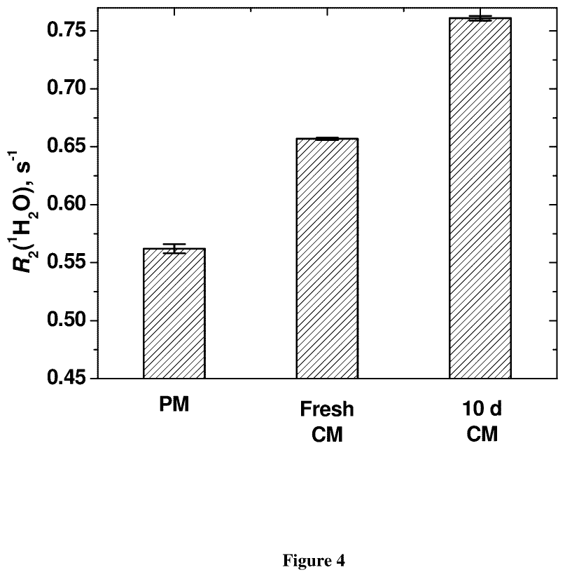

FIG. 4 compares the results of R₂(¹H₂O) measurements for three cell media-fresh propagation medium (PM), fresh conditioned medium (Fresh CM), and 10 days old conditioned medium (10d CM). It is evident that the longer a cell culture stays in a medium, the greater the water relaxation rate. Without being bound by theory, it is assumed that such increase results from the gradual accumulation of the various metabolites excreted by cells as well as dead cell debris during their growth and aging. Hence, wNMR technology, when applied to cell-free culture media, could be used for indirect monitoring of the cells' lifecycle, e.g., in a bioreactor.

### Example 6. Monitoring Cell Growth by wNMR

The sensitivity of wNMR to cell growth in a cell culture was also analyzed by monitoring the dynamic changes of R₂(¹H₂O) during the cell culture lifecycle. The protocol described below was followed, and the R₂(¹H₂O) measured on Days 0, 1, 2, 3, 6, 7, 8, 9, 10, and 14 simultaneously correlating the results with the cell count measurements conducted in parallel.

The CHO cell culture process started with a seed train lasting 4 days. The cells were then passaged and inoculated at an initial concentration in Fusion media, cultured in a flask. Following the seed train, a 14-day fed-batch culture was initiated on day 4 by passing cells into a new CHO Fed Batch Media. The culture was maintained until cell count reached an amount (generally, ca. 3×10⁶ cells per mL). Throughout the fed-batch phase, cells were fed with 40% glucose and 5% of the starting volume of Feed #1 and Feed #4 media on alternate days.

Seed Train. CHO cells were initially at 1.3×10⁶ cells per mL. Cells were transferred to a 125 mL flask containing 30 mL of CHO Fusion media. This dilution resulted in an initial cell count of approximately 0.3×10⁶ cells per mL. Cells were allowed to grow until they reached a density of about 3×10⁶ cells per mL.

Day 0 (Start of Fed-Batch Culture). After the seed train completion, cells were transferred to a new 125 mL flask. The new flask contains 30 mL of CHO Fed Batch Media. Cells are seeded at a density of ca. 0.5×10⁶ cells per mL. They were again allowed to grow until they reached a density of ca. 3×10⁶ cells per mL.

Feeding Schedule (Days 3, 5, 7, 9, 11 and 13). On these days, the culture was supplemented with 40% glucose solution, added to reach a final glucose concentration of about 4 g/L. A mixture of CHO Feed #1 and Feed #4 media added 5% of the starting culture volume.

Cell growth and productivity were monitored every 24 hours, with 0.6 mL samples analyzed for cell count and viability. All cell counts were performed in accordance with the protocols described in Example 1 using Beckman Coulter Vi-CELL automated cell counter/analyzer. In all measurements, the samples contained >95% viable cells.

In this experiment, the above standard protocol was changed on Day 7, wherein the aliquot of the cell culture was transferred to new flask and fresh feeding media was added. Therefore, the initial cell count on the Day 8 after this dilution was 0.8×10⁶ cells per mL.

For wNMR measurements, 2 mL aliquots of each sample taken on the Days 0, 1, 2, 3, 6, 7, 8, 9, 10, and 14 were placed in ISO 2R sterile glass vials with sterile rubber septa under sterile conditions in the biosafety cabinet. All measurements of water proton transverse relaxation were performed as described above in the Example 2. Parameters of water proton relaxation were measured using the same CPMG approach as disclosed in the Example 2 herein. However, in addition to the measurements at the interpulse delay τ=0.5 ms, the values of R₂(¹H₂O) at τ=3.0 ms and τ=5.0 ms were also measured. Data processing was also the same as in the Example 2 hereinabove.

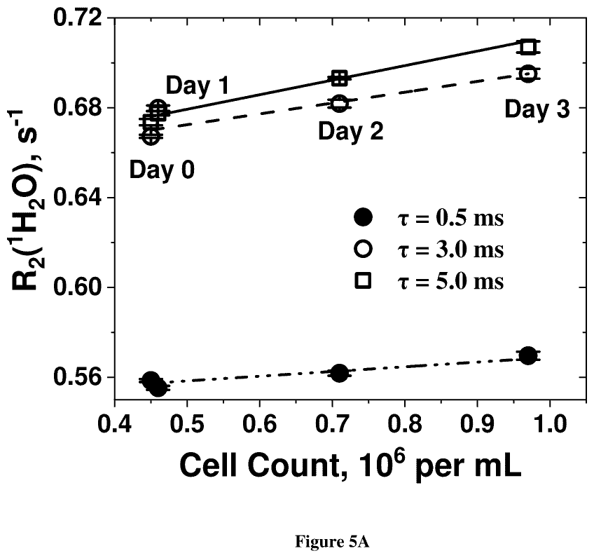

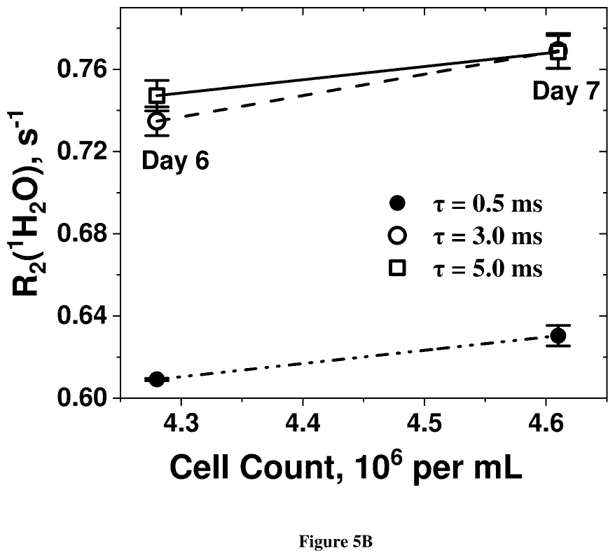

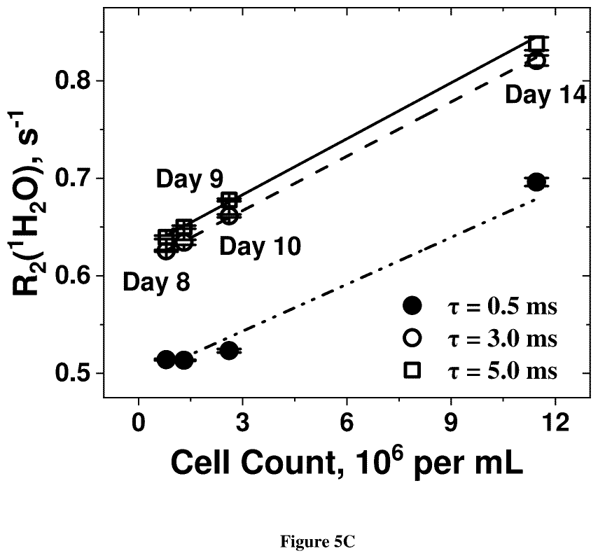

The dependences of the water proton transverse relaxation rate R₂(¹H₂O) on the cell counts (viability >95%) during the fed-batch phase of the culture lifecycle (Days 0-3) is shown in FIG. 5A. These dependences were observed for three different τ values: for τ=0.5 ms with the slope 0.021 s⁻¹/(10⁶ cells per mL) at linear fit quality <r₂>=0.808; for t=3 ms with the slope 0.049 s⁻¹/(10⁶ cells per mL) at <r₂>=0.675; and for t=5 ms with the slope 0.065 s⁻¹/(10⁶ cells per mL) at <r₂>=0.986. Here, even at the somewhat low cell counts at the early fed-batch phase, these linear dependences could be used to provide reliable estimates of the cell counts from the R₂(¹H₂O) data.

FIG. 5B shows the growth of the cell count (viability >95%) and R₂(¹H₂O) during two days (Days 6 and 7) after the feeding schedule started on Day 3. These results demonstrate that even the small changes in the cell count (from ca. 4.3×10⁶ to ca. 4.6×10⁶ cells per mL) could be reliably traced by wNMR.

The dependences of the water proton transverse relaxation rate R₂(¹H₂O) on the cell counts (viability >95%) during the feeding schedule phase (Days 8-10, and 14) is shown in FIG. 5C. Since on Day 7 the aliquot of the cell culture was transferred to a new flask and fresh feeding media was added, the initial cell count on the Day 8 after this dilution was only 0.8×10⁶ cells per mL. Almost perfect linear fits at different interpulse delay values: slope 0.016 s⁻¹/(10⁶ cells per mL), <r₂>=0.923 (τ=3 ms); slope 0.018 s⁻¹/(10⁶ cells per mL), <r₂>=0.997 (τ=3 ms); and slope 0.019/(10⁶ cells per mL), <r₂=0.999 (τ=5 ms) demonstrate strict correlations of wNMR and cell counts data. Therefore, after proper calibration, these linear dependences of R₂(¹H₂O) vs. cell counts could be advantageously used to obtain of the cell counts during the final phase of the feeding and maturation of the cell culture from the wNMR data.

It should be pointed out that the data collected using three different values of the interpulse delay τ show that larger τ-values provide much greater absolute values of the water proton transverse relaxation rate R₂(¹H₂O) (FIGS. 5A-5C). Hence, it should be appreciated by the person of ordinary skill in the art that higher dynamic range of the R₂(¹H₂O) values attained at larger τ could provide much greater sensitivity of wNMR when monitoring changes in the total cell counts in the process of cell culture growth.

Although the invention has been disclosed herein in details with reference to various embodiments and features, it will be appreciated by a person with ordinary skill in the art that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

What is claimed is:

1. A method of estimating a cell count in an aliquot comprising cells, said method comprising measuring the relaxation rate of solvent R in the aliquot comprising cells, and comparing the measured R of solvent to a calibration curve to estimate the cell count of the aliquot comprising cells,
wherein the R is selected from the transverse relaxation rate of solvent R₂ or the longitudinal relaxation rate of solvent R₁.

2. The method of claim 1, wherein the solvent comprises water.

3. The method of claim 1, wherein the aliquot comprising cells is a cell culture, a growing cell culture, a sample associated with the evaluation of the effects of a drug candidate on cells, a sample of thawed previously cryopreserved cells, a sample comprising a resuspended cell pellet, or a cell sample aliquoted from a bioreactor.

4. The method of claim 1, wherein the aliquot is contained in a vial or passes through a conduit communicatively connected to a bioreactor.

5. The method of claim 4, wherein the relaxation rate of solvent R is measured without adding any additives to the vial containing the aliquot.

6. The method of claim 1, wherein the relaxation rate of solvent R is measured using nuclear magnetic resonance (NMR) or magnetic resonance imaging (MRI).

7. The method of claim 1, wherein the calibration curve is prepared by plotting relaxation rate R versus a known cell count of at least one calibration sample.

8. The method of claim 7, wherein the at least one calibration sample and the aliquot comprising cells were cultured under substantially identical conditions.

9. The method of claim 1, wherein following measurement of the relaxation rate of solvent R, the aliquot comprising the cells is not altered and can continue to be used.

10. The method of claim 1, wherein R is the transverse relaxation rate of solvent R₂.

11. A method of estimating a percent cell viability in an aliquot comprising cells, said method comprising measuring the relaxation rate of solvent R in the aliquot comprising cells, and comparing the measured R of solvent to a calibration curve to estimate the percent cell viability of the aliquot comprising cells,
wherein the R is selected from the transverse relaxation rate of solvent R₂ or the longitudinal relaxation rate of solvent R₁.

12. The method of claim 11, wherein the solvent comprises water.

13. The method of claim 11, wherein the aliquot comprising cells is a cell culture, a growing cell culture, a sample associated with the evaluation of the effects of a drug candidate on cells, a sample of thawed previously cryopreserved cells, a sample comprising a resuspended cell pellet, or a cell sample aliquoted from a bioreactor.

14. The method of claim 11, wherein the aliquot is contained in a vial or passes through a conduit communicatively connected to a bioreactor.

15. The method of claim 14, wherein the relaxation rate of solvent R is measured without adding any additives to the vial containing the aliquot.

16. The method of claim 11, wherein the relaxation rate of solvent R is measured using nuclear magnetic resonance (NMR) or magnetic resonance imaging (MRI).

17. The method of claim 11, wherein the calibration curve is prepared by plotting relaxation rate R versus a known percent cell viability of at least one calibration sample.

18. The method of claim 17, wherein the at least one calibration sample and the aliquot comprising cells were cultured under substantially identical conditions.

19. The method of claim 11, wherein following measurement of the relaxation rate of solvent R, the aliquot comprising the cells is not altered and can continue to be used.

20. The method of claim 11, wherein R is the transverse relaxation rate of solvent R₂.
